(19) United States
(12) Patent Application Publication
(10) Pub. No.: US 2008/0215041 A1
(43) Pub. Date: Sep. 4, 2008

(54) Cannula/optical fibre assembly and laser instrument including said assembly

(75) Inventors: Jaouad Zemmouri, Genech (FR); Jean Ringot, Mons-En-Baroeul (FR)

Correspondence Address: HARNESS, DICKEY & PIERCE, P.L.C., P.O. BOX 828, BLOOMFIELD HILLS, MI, 48303, US

(73) Assignee: Optical System & Research For Industry And Science Osyris SA, Hellemmes (FR)

(21) Appl. No.: 12/074,216

(22) Filed: Feb. 29, 2008

(30) Foreign Application Priority Data
Mar. 2, 2007 (FR) 07/01513

(51) Int. Cl. A61B 18/22

(52) U.S. Cl. 606/15

(57) ABSTRACT

The assembly includes a cannula, which comprises an opening at a so-called distal extremity and electromagnetic radiation wave guiding means, which include an optical fibre introduced inside the cannula and which enable the electromagnetic radiation to be guided to the opening of the distal extremity of the cannula, in such a way that this electromagnetic radiation is emitted frontally by said distal opening of the cannula. The external diameter of the protective sheath of the optical fibre is essentially equal to the internal diameter of the cannula in at least one distal portion of said cannula; the core of the optical fibre is stripped on a distal part of the fibre, and the stripped distal part of the optical fibre is accommodated entirely inside the cannula.

### FIELD OF THE INVENTION

The present invention relates to a new assembly comprising an optical fibre introduced inside a cannula and the use of said cannula/optical fibre assembly to make a laser instrument intended for use in the field of medicine.

### PRIOR ART

Needles containing an optical fibre are commonly used in the field of medicine, the optical fibre enabling a laser beam to be transmitted from a laser energy source, for treating or diagnosing diseases. This type of assembly, using a laser energy source, can be used in numerous medical fields, in particular for the treatment of varicose veins, the treatment of adiposis, for percutaneous diagnostics or else in the field of ophthalmic surgery. There are thus multiple applications for this type of assembly as well as multiple constraints linked to each application.

Currently, for instance, in the case of the treatment and reduction of adiposis or subcutaneous adipose cells, the liposuction technique is used, which consists of introducing a probe with a diameter of approximately 5 mm underneath the skin of a patient and of suctioning out fat. It is also common to use an ultrasound probe introduced under the skin of a patient to destroy the membrane of the adipose cells. In the latter case, when the membrane is destroyed, a liquid escapes and must also be suctioned out. These two techniques present the disadvantages of a lack of homogeneity at the level of the treated zones and of causing bleeding of the patient, in particular due to the size of the perforations carried out in the patient's skin.

For these reasons, a new technique and a new device, described for instance in document U.S. Pat. No. 6,206,873, have appeared. The device comprises an optical fibre positioned inside a cannula, which is coupled at its proximal extremity to a laser energy source and which enables a laser beam (electromagnetic radiation) to be emitted to its distal extremity. The cannula consists of a hollow needle, the distal extremity of which is open and bevelled, and reveals the distal extremity of the optical fibre.

The technique consists in piercing the skin of the patient, in introducing the needle into a subcutaneous layer of adipose cells of the patient and in radiating said layer of adipose cells by means of the laser beam. The irradiation with the laser beam triggers the lipolysis of the adipose layer and results in the rupture of the membrane of the adipose cells, thus transforming the cells into a liquid substance. This liquid substance can be suctioned out or is preferably left as it is to be drained by the lymphatic system or by phagocytic action. The technique described in this document enables a uniform treatment to be carried out of a layer of adipose cells, while also eliminating the problems of the patient bleeding and reducing the size of the perforations in the patient's skin. The bleeding of the patient is in fact eliminated thanks to the use of the energy of the laser beam for cauterizing the blood vessels.

The needle/optical fibre assembly described in this U.S. Pat. No. 6,206,873 publication presents the following disadvantages.

A first disadvantage arises from the fact that the distal extremity of the optical fibre extends beyond the distal opening of the needle. The result is a risk of the optical fibre rupturing upon the device being introduced into the human body, something that is very detrimental and can prove to be a danger to the patient's health.

Usually, an optical fibre includes three concentric parts:
a central part, for instance made of Si0₂ (which may or may not be doped), ensuring the guided propagation of an electromagnetic radiation,
a very thin intermediate layer (for instance made of Si0₂ or of polymer), generally called “cladding”, surrounding the central part and displaying a different refractive index to the central part, so as to confine the electromagnetic radiation to the central part,
a mechanical polymer protective sheath, which is thicker.

In the present text, including the claims, the term “core” designates the central part and the intermediate layer (“cladding”) of the optical fibre, and the term “sheath” designates the above-mentioned mechanical protective sheath.

In order to improve the emission of the laser beam and to avoid burning of the sheath surrounding the core of the optical fibre, it is preferable in practice to strip the core of the optical fibre at its distal extremity. The core of the optical fibre thus stripped is no longer protected at the distal extremity of the fibre by the mechanical protective sheath surrounding the core of the fibre. The core of the optical fibre, however, is very fragile from a mechanical point of view, which significantly increases the risks of rupture.

A second disadvantage is linked to the immobilisation of the optical fibre with relation to the needle. In the device of the above-mentioned publication U.S. Pat. No. 6,206,873, the optical fibre is immobilised with relation to the needle by means of a mechanical clamping system including an elastic ring, through which the optical fibre is passed, and a clamping screw. This mechanical clamping system does not enable the optical fibre to be reliably immobilised with relation to the needle. The result is that, in practice, during the implementation of this device, there is a significant risk of the optical fibre sliding with relation to the needle, which increases the risks of rupture of the optical fibre inside the patient's body.

The third disadvantage is linked to the presence, between the optical fibre and the needle, of a gap enabling tissues to penetrate inside the needle. Upon implementing the laser, however, the tissues that penetrate between the needle and the optical fibre are burnt by the laser. On the one hand, all or part of these burnt tissues can, in a detrimental manner, end up inside the patient's body; on the other hand, they can cause damage to the optical fibre.

The French patent application FR-A-2 875 122 describes a medical instrument capable of being used in vascular occlusion and comprising, in one embodiment, an optical fibre positioned inside a needle. This needle/optical fibre embodiment presents the three disadvantages described above in relation to the device of publication U.S. Pat. No. 6,206,873.

The document FR-A-2 875 122 suggests another solution in which the optical fibre is replaced by a silica electromagnetic radiation wave guide that is integral with the needle; more particularly, this electromagnetic radiation wave guide is formed by an internal “covering” of the inside of the needle. The technical concept of “covering” is not clarified in this document and is not clear. Nevertheless, the realisation of such an electromagnetic radiation wave guide inside a needle appears to be difficult to carry out; in addition, since to date no such device has been marketed, its effectiveness can be questioned.

American patent application US 2006/0078265 describes a device primarily consisting of a laser energy source linked to a hand piece and used to project a laser beam on human tissues. The hand piece comprises in particular an optical fibre and a cannula containing the optical fibre. The distal part of the optical fibre is in this case entirely encased in the cannula. The cannula is closed at its distal extremity and a lateral opening is arranged in the cannula for a lateral diffusion of the laser beam.

The protective sheath of the optical fibre has been partially removed on a distal portion of the fibre, thus revealing the core of the optical fibre. The distal extremity of the sheath is abutted against three contact points, distributed on the periphery of the internal wall of the cannula, which enables the distal extremity of the optical fibre to be correctly positioned with relation to the lateral opening of the cannula enabling the laser beam to be diffused.

In the devices described in this publication US 2006/0078265, the problem of immobilising and maintaining the optical fibre with relation to the cannula is not resolved. Thus, despite the fact that the extremity of the optical fibre is in abutment against internal contact points, the optical fibre can slide backwards; upon implementing this device, if the optical fibre is not correctly immobilised and maintained in its distal part, there is a risk of rupturing the core of the fibre at its stripped distal extremity.

In addition, as in the other documents described above, there is a gap between the optical fibre and the cannula enabling tissues to penetrate.

Finally, in all the devices described in this publication US 2006/0078265, the laser emission is lateral and is not frontal, and it is essential that a reflector be placed inside the needle to deflect the laser beam and to diffuse it through the lateral opening of the cannula. The implementation of the reflector complicates the manufacture of the device in a detrimental manner.

The publication US 2002/0138073 discloses a surgical laser probe implementing an optical fibre, the emission distal part of which is mounted inside a cylindrical tip. The end of the cylindrical tip is closed by a mirror and the cylindrical wall of this tip is made of a material that is transparent to the laser beam delivered at fibre's end. This cylindrical tip allows a lateral diffusion of the electromagnetic radiation delivered by the optical fibre but doesn't allow a frontal emission. Further, in order to obtain this lateral diffusion of the electromagnetic radiation, a scattering medium is placed inside the cylindrical tip between the distal end of the optical fibre and the closed end of the tip.

The publication U.S. Pat. No. 5,269,777 discloses a surgical laser probe implementing an optical fibre, the emission distal part of which is inserted inside a scattering tip made of silicone having preferably a length comprised between 0.5 cm and 5 cm. This scattering tip is fastened on the distal end of the fibre core, and doesn't constitute a cannula. After fastening of this tip, a second internal scattering layer is deposited inside the tip, for example by injection moulding. This internal scattering layer contains scattering particles such as for example diamond dust, TiO₂ or alumina. The electromagnetic radiation, which is guided by the optical fibre to its distal end, is not guided anymore inside the scattering tip, but is scattered in any directions by the internal scattering layer of the tip.

### OBJECT OF THE INVENTION

According to a first aspect, the main object of the present invention is to provide a new cannula/optical fibre assembly, which enables a frontal emission of an electromagnetic radiation, which enables the risk of rupture of the optical fibre to be reduced and which enables the optical fibre to be reliably immobilised with relation to the cannula.

### SUMMARY OF THE INVENTION

According to this first aspect of the invention, the optical fibre/cannula assembly includes a cannula, which comprises an opening at a so-called distal extremity, and electromagnetic radiation wave guiding means. These electromagnetic radiation wave guiding means include an optical fibre introduced inside the cannula, and enable an electromagnetic radiation to be guided to the opening of the distal extremity of the cannula, in such a way that this electromagnetic radiation is emitted frontally by said cannula's distal extremity. Said optical fibre includes a core surrounded by an external protective sheath, the external diameter (D₁) of the protective sheath of the optical fibre being essentially equal to the internal diameter (d₂) of the cannula in at least one distal portion of said cannula. The core of the optical fibre is stripped on a distal part of the fibre, and the stripped distal part of the optical fibre is accommodated entirely inside the cannula.

The term “essentially equal” means that said external diameter of the sheath of the optical fibre is very slightly inferior to the internal diameter of the cannula, with a functional clearance between the sheath of the optical fibre and the cannula, which is just sufficient to enable the optical fibre to slide inside the cannula during the operation of introducing the fibre.

In the present text, the term “cannula” is given in its general sense and covers any hollow support, whether elongate, curved or straight, enabling the optical fibre to be introduced and guided inside the human body. The cannula can in particular include a hollow tube, the distal extremity of which is rounded and non-aggressive to tissues; the cannula can also include a hollow needle, the distal extremity of which is bevelled and enables tissues to be pierced.

According to a second aspect of the invention, capable of being implemented independently of the first above-mentioned aspect, the assembly of the invention includes a cannula and electromagnetic radiation wave guiding means comprising an optical fibre, which is introduced inside the cannula; the cannula is crimped onto the optical fibre. According to this second aspect, the cannula can either be open at its distal extremity or closed at its distal extremity and can include in its distal part a lateral opening for the emission of the electromagnetic radiation, in a manner comparable to the cannula/optical fibre assembly described in publication US 2006/0078265.

According to a third aspect of the invention, capable of being implemented independently of the above-mentioned first and second aspects, the assembly of the invention includes a cannula, which is open at its so-called distal extremity, and electromagnetic radiation wave guiding means, which include an optical fibre introduced inside the cannula and which enable the electromagnetic radiation to be guided to the open distal extremity of the cannula, said optical fibre including a core surrounded by an external protective sheath. The core of the optical fibre is stripped at the distal extremity of the fibre; the distal part of the optical fibre is accommodated entirely inside the cannula and the distal extremity of the optical fibre blocks the distal opening of the cannula.

Preferably, but optionally according to the invention, for the above-mentioned three aspects, the cannula/optical fibre assembly includes the following additional technical characteristics, taken individually or in combination:
the cannula includes in its distal part a hollow insert, which includes a continuous cavity, and the stripped distal part of the core of the fibre is threaded into this hollow insert;
the sheath of the optical fibre is positioned in abutment inside the cannula; more particularly, the sheath of the optical fibre is positioned in abutment against the insert;
the stripped distal part of the core of the fibre is accommodated entirely inside the hollow insert;
the hollow insert is made out of a thermally conductive material and enables the sheath of the optical fibre to be thermally protected;
the distal extremity of the core of the optical fibre is flush with the distal opening of the cannula;
the stripped distal part of the optical fibre blocks the distal opening of the cannula, which advantageously prevents the penetration of foreign bodies into the cannula;
the electromagnetic radiation wave guiding means include an additional optical wave guide fixed to the cannula and the distal extremity of the core of the optical fibre is preferably in contact with said additional optical wave guide;
the additional optical wave guide is flush with the distal opening of the cannula;
the additional optical wave guide blocks the distal opening of the cannula, which advantageously prevents the penetration of foreign bodies into the cannula;
the cannula is crimped onto the sheath of the optical fibre, which enables the optical fibre to be simply and efficiently immobilised with relation to the cannula.

Another object of the invention is a laser instrument including a laser source coupled to the optical fibre of an assembly of the invention, said assembly being able to conform to the first, second or third above-mentioned aspects.

### BRIEF DESCRIPTION OF THE DRAWINGS

The invention and its advantages will be more easily understood upon reading the following description of a non-limiting and non-exhaustive example only and with reference to the accompanying drawings, in which:

FIG. 1 shows a laser instrument according to the invention;

FIG. 2 shows a cross section of a cannula/optical fibre assembly in a first embodiment of the invention;

FIG. 3 shows a cross section of a cannula/optical fibre assembly in a second embodiment of the invention;

FIG. 4 shows a cross section of a cannula/optical fibre assembly in a third embodiment of the invention;

FIG. 5 shows a cross section of a cannula/optical fibre assembly in a fourth embodiment of the invention;

FIG. 6 shows a cross section of a cannula/optical fibre assembly in a fifth embodiment of the invention;

FIG. 7 shows a cross section of a cannula/optical fibre assembly in a sixth embodiment of the invention;

FIGS. 8 to 11 show cross sections of a cannula/optical fibre assembly according to seventh, eighth, ninth and tenth embodiments of the invention;

FIGS. 12 and 13 show an optical fibre in a cannula resulting from a first step of a first method of assembly;

FIGS. 14 and 15 show an optical fibre in a cannula resulting from a second step of said first method of assembly;

FIGS. 16 and 17 show an optical fibre in a cannula resulting from a third step of said first method of assembly;

FIGS. 18 and 19 show an optical fibre in a cannula resulting from a first step of a second method of assembly;

FIGS. 20 and 21 show an optical fibre in a cannula resulting from a second step of said second method of assembly.

### DETAILED DESCRIPTION

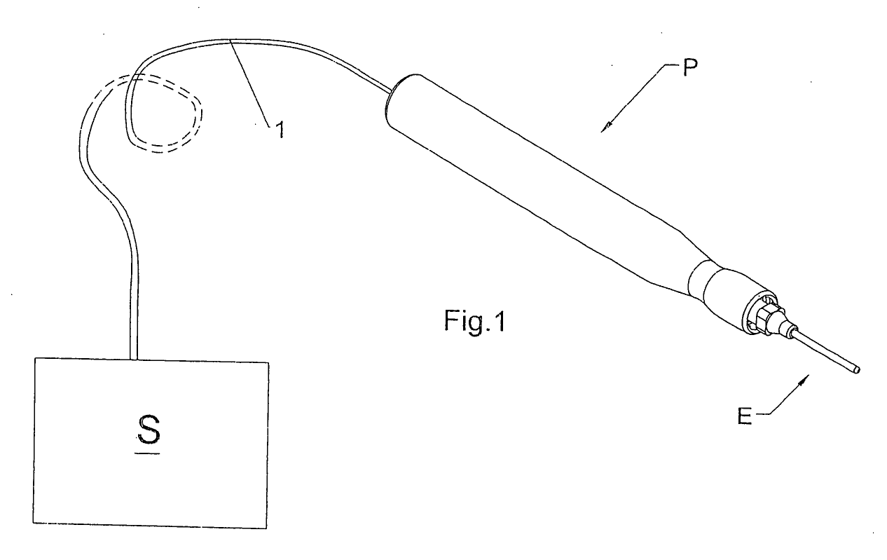

With reference to FIG. 1, a laser instrument according to the invention includes a cannula/optical fibre assembly with reference designation E, on which a hand piece P is adapted, and a laser source S, which is coupled to the optical fibre of the assembly E. The hand piece P is known as such and facilitates the handling and manipulation of the cannula/optical fibre assembly E. This laser instrument is intended to be used in the medical field, for any type of laser treatment, and for instance and in a non-limiting and non-exhaustive manner, for the treatment of varicose veins, for the treatment of adiposis, for percutaneous diagnostics or else in the field of ophthalmic surgery. It is up to the person skilled in the art to select the laser source best adapted to the uses of the laser instrument.

Various embodiments of the cannula/optical fibre assembly E will be described in detail hereinafter.

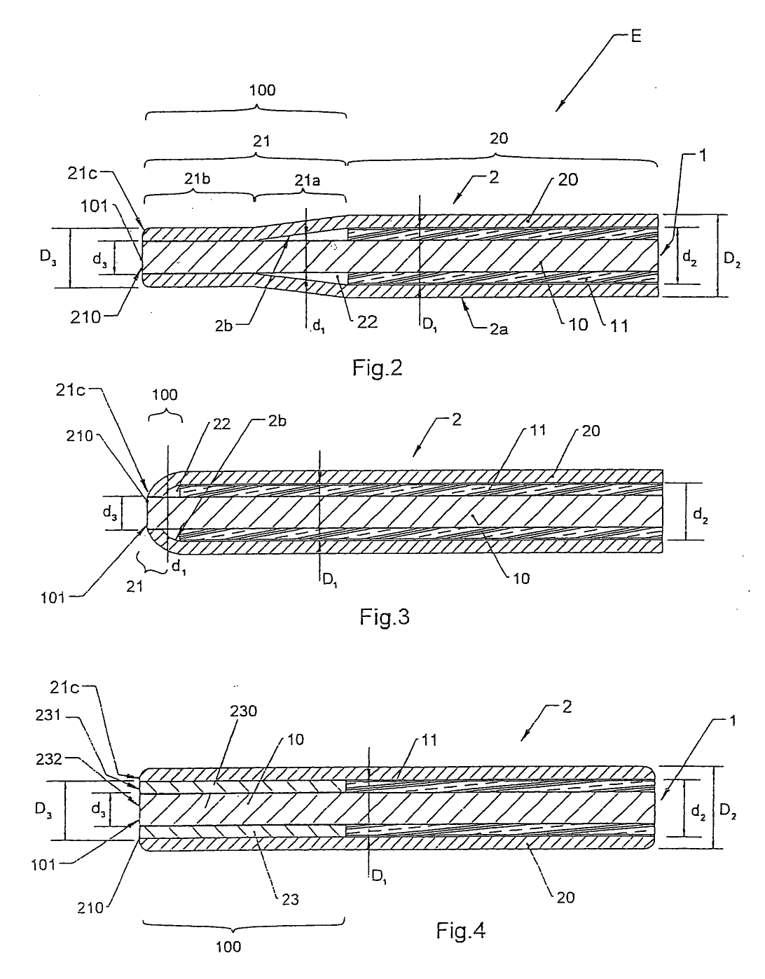

In a first embodiment shown in FIG. 2, the assembly E comprises an optical fibre 1, which enables the electromagnetic radiation (light) supplied by the laser source S to be guided by total internal reflection, and the distal part of which (opposite to the laser source) is introduced inside a cannula 2. In FIG. 2, only the distal portion of the optical fibre 1 introduced inside the cannula 2 and a distal part of the cannula 2 are represented. In addition, the length of the cannula 2 can be increased or decreased according to its application.

The optical fibre 1 is known as such and comprises a core 10 for the guided propagation of the electromagnetic radiation supplied by the laser source and a mechanical protective sheath 11, for instance made of plastic, surrounding the core 10. As mentioned previously, the core of the optical fibre includes a central part for the guided propagation of the electromagnetic radiation and a thin intermediate layer, commonly called “cladding”, and surrounding the central part, for the confinement of the electromagnetic radiation in the central part. On the annexed drawings, said central part and said thin intermediate layer, which form the core of the optical fibre, are not differentiated.

The optical fibre has a diameter D₁ (external diameter of the sheath 11) and the core 10 of the fibre 1 has a diameter d₁. The distal part 100 of the core 10 of the fibre 1 is stripped, namely the protective sheath 11 does not cover the core 10 and thus reveals the distal part 100 of the core 10. The risks of burning the sheath 11 by the laser beam emitted at the distal extremity 101 of the core 10 are thus avoided.

The cannula 2 consists of a flexible tube which is opaque to the electromagnetic radiation emitted by the laser source S, for instance a flexible stainless steel tube. In the particular example illustrated, it comprises a hollow cylindrical body 20 extended by a hollow distal part 21 essentially in the shape of a funnel, said hollow body 20 and the hollow distal part 21 defining a continuous internal cavity 22. In the remainder of the description, the external face of the cannula 2 will be given the reference number 2a, and the internal face of the cannula 2 will be given the reference number 2b.

In the remainder of the description, the internal diameter of the cavity 22 in the part of the cannula 2 corresponding to the hollow cylindrical body 20 is given the reference d₂, and the external diameter of the cannula 2, at the level of the hollow cylindrical body 20 is given the reference D₂.

The above-mentioned distal part 21 of the cannula 2 comprises a first part 21a essentially in the shape of a truncated cone, which extends the cylindrical body 20 of the cannula 2, and the transversal section of which decreases in the direction of the second cylindrical part 21b, of a smaller diameter than the cylindrical body 20. The distal extremity 21c of the distal part 21 of the cannula 2 is open (distal opening 210 aligned on the longitudinal axis of the cannula 2). In the particular example of FIG. 2, the diameter of the distal opening 210 of the cannula 2 is equal to the diameter of the cavity 22 of the cannula at the level of the above-mentioned second part 21b, and is given the reference d₃ in the remainder of the description. The external diameter of the cannula 2 at the level of the second part 21b is given the reference D₃.

Advantageously, the distal extremity 21c of the cannula 2 is rounded to limit the risks of tearing the tissues of a patient during the introduction of the assembly E.

The external diameter D₁ of the sheath 11 of the fibre 1 is essentially equal to the internal diameter d₂ of the cavity 22. The term “essentially equal” means that the external diameter D₁ of the protective sheath 11 of the optical fibre 1 is very slightly inferior to the internal diameter d₂ of the cannula 2, with a functional clearance between the sheath 11 of the optical fibre 1 and the cannula 2, which is just sufficient to enable the optical fibre 1 to slide inside the cannula 2 during the operation of introducing the fibre. Typically, the difference in diameter (d₂−D₁) is inferior to 100 μm and preferably inferior to 50 μm. The result is on the one hand that the distal part of the optical fibre 1 is maintained in a reliable manner inside the cannula 2. On the other hand, it is easy to reliably immobilise in translation the optical fibre 1 with relation to the cannula 2, by a localised clamping of the cannula 2 on the sheath 11 of the fibre 1 and, for instance, as shall be detailed hereinafter, by a simple crimping of the cannula 2 on the sheath 11 of the optical fibre 1.

Also, with reference to FIG. 2, the stripped distal part 100 of the optical fibre 1 is accommodated entirely inside the cannula 2.

More particularly, the protective sheath 11 is abutted inside the cannula 2 against the internal face 2b of the first part in the shape of a truncated cone 21a.

More particularly, as illustrated on FIG. 2, the distal extremity 101 of the stripped core 10 of the optical fibre is flush with the distal opening 210 of the cannula 2.

During the introduction and the manipulation of the assembly E (cannula/fibre) into the human body, the risks of breaking the stripped distal part 100 of the core 10 of the optical fibre 1 are thus advantageously avoided, said stripped distal part 100 being more fragile from a mechanical point of view.

More particularly still, in the example of FIG. 2, the diameter d₁ of the core 10 of the fibre is essentially equal to the diameter d₃ of the distal opening 210 of the cannula 2. The core of the stripped fibre 10 thus blocks the distal opening 210 of the cannula 2 and advantageously prevents the penetration of foreign bodies into the cannula 2, and in particular the penetration of tissues during the movement of the cannula 2 and the optical fibre 1 in the human body.

More particularly, but unnecessarily, at the level of the distal opening 210 of the cannula 2, the distal extremity of the fibre's core may be glued inside the distal extremity 21b of the cannula, by mean for example of a glue which can be activated by UV radiation. This glue enables to ensure, between the stripped fibre's core and the cannula's internal wall, a perfect tightness preventing any penetrations by the distal opening 210 of foreign body inside the cannula 2.

In operation, the entire electromagnetic radiation emitted by the laser source S is guided by total internal reflections by the optical fibre 1 to the distal opening 210 of the cannula 2, and the totality of this electromagnetic radiation is emitted frontally by this distal opening 210 of the cannula 2.

In a second embodiment of the invention, shown in FIG. 3, the distal part 21 of the cannula 2 is essentially hemispherical. This essentially hemispherical rounded portion of the distal part 21 makes it possible to go from a diameter d₂ of the cavity 22 to a smaller diameter d₃. The protective sheath 11 is thus abutted inside the cannula 2 against the internal wall 2b of the distal hemispherical part 21. In this embodiment, in a manner similar to the first embodiment of FIG. 2, the stripped distal part 100 of the core 10 of the optical fibre is accommodated entirely inside the cannula 2, and the distal extremity 101 of the core 10 of the fibre 1 is flush with and blocks the distal opening 210 of the cannula 2.

In a third embodiment of the invention, shown in FIG. 4, the cannula 2 comprises a hollow cylindrical body 20 with an external diameter D₂ and an internal diameter d₂, and a hollow cylindrical insert 23. The insert 23 comprises a continuous internal cavity 230, and is fixed to the inside of the hollow cylindrical body 20.

In this embodiment, this insert 23 is more particularly made out of a thermally conductive material, thus enabling the heat produced by the laser beam to be evacuated in the direction of the cannula 2, and a propagation of said heat to be avoided in the direction of the sheath 11 of the optical fibre 1. Obviously, the insert 23 must be made out of a material resistant to the heat produced by the laser. In addition, this insert 23 acts as a mechanical abutment for the protective sheath 11 of the optical fibre 1, and thus facilitates the positioning of the optical fibre in the cannula 2. The insert 23 is for instance made out of metal, particularly stainless steel, and is welded inside the hollow cylindrical body 20, by laser, by brazing or by gluing.

More particularly, the insert 23 has an external diameter D₃ and an internal diameter d₃ and is accommodated entirely inside the hollow cylindrical body 20, such that the distal extremity 231 of said insert 23 is flush with the distal opening 210 of the hollow body 20. The stripped distal part 100 of the core 10 of the optical fibre 1 is accommodated entirely inside the insert 23, the protective sheath 22 of the fibre 1 being in abutment inside the hollow body 20 against the insert 23. More particularly, the distal extremity 101 of the core 10 of the fibre 1 is flush with and blocks the opening 232 of the distal extremity 231 of the insert 23.

In this embodiment, the external diameter D₃ of the insert 23 is essentially equal to the diameter d₂ of the cavity 22. Similarly, the internal diameter d₃ of the insert is essentially equal to the diameter d₁ of the core 10 of the fibre 1. It is obviously necessary to respect certain tolerance clearances to enable the stripped core 10 of the fibre to be introduced into the cavity 230 of the insert 23, and the insert 23 to be introduced into the cavity 22 defined by the hollow body 20.

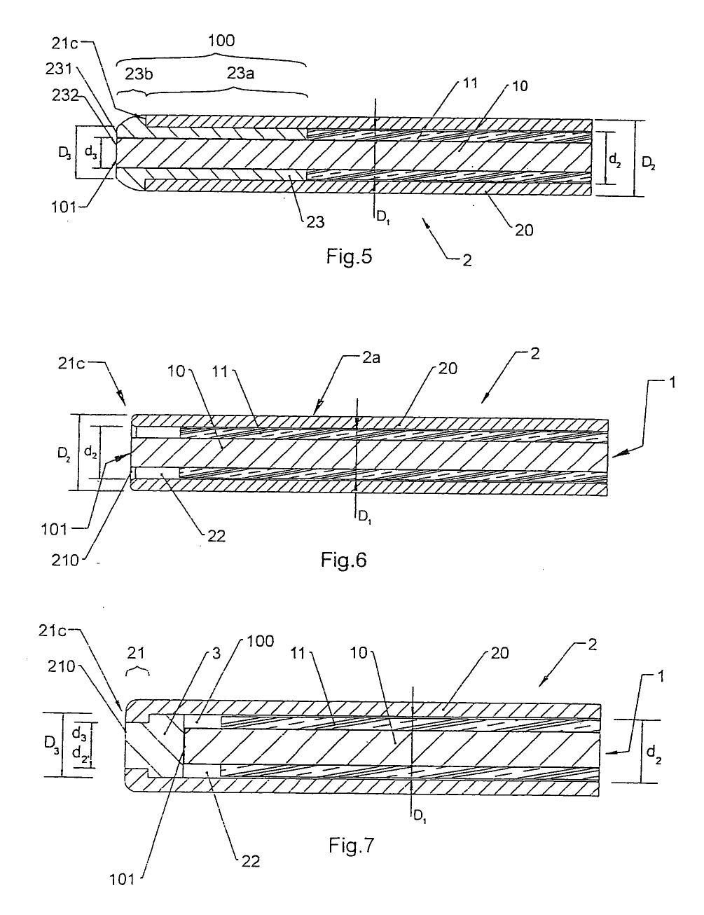

FIG. 5 shows a fourth embodiment of the invention, which differs from the embodiment of FIG. 4 due to the implementation of an insert 23 of a different shape. This hollow insert 23 includes a first cylindrical part 23a and a second part 23b, having a larger transversal section and called head of the insert 23 hereinafter. The insert 23 is fixed to the hollow cylindrical body 20 of the cannula 2, such that, on the one hand, the tubular part 23a of the insert 23 is accommodated entirely inside the body 20 and, on the other hand, the head 23b of the insert 23 is in abutment against the distal extremity face 21c of the hollow body 20. In this embodiment, the stripped distal extremity 101 of the core 10 of the optical fibre juts out with relation to the distal extremity face 21c of the hollow body 20, but is accommodated entirely inside the insert 23 and is flush with and blocks the distal opening 232 of the head 23b of the insert 23.

In a fifth embodiment of the invention shown in FIG. 6, the cannula 2 consists of a hollow cylindrical body 20 with an external diameter D₂ and with an internal diameter d₂. The optical fibre 1, of an external diameter D₁, is positioned inside the cavity 22 of the cannula 2 such that the stripped distal extremity 101 of the core of the fibre 1 is flush with the distal opening 210 of the cannula 2, without however blocking this distal opening 210.

In order to manufacture the assemblies E (cannula/optical fibre) of FIGS. 2 to 6, two different manufacturing procedures can be implemented.

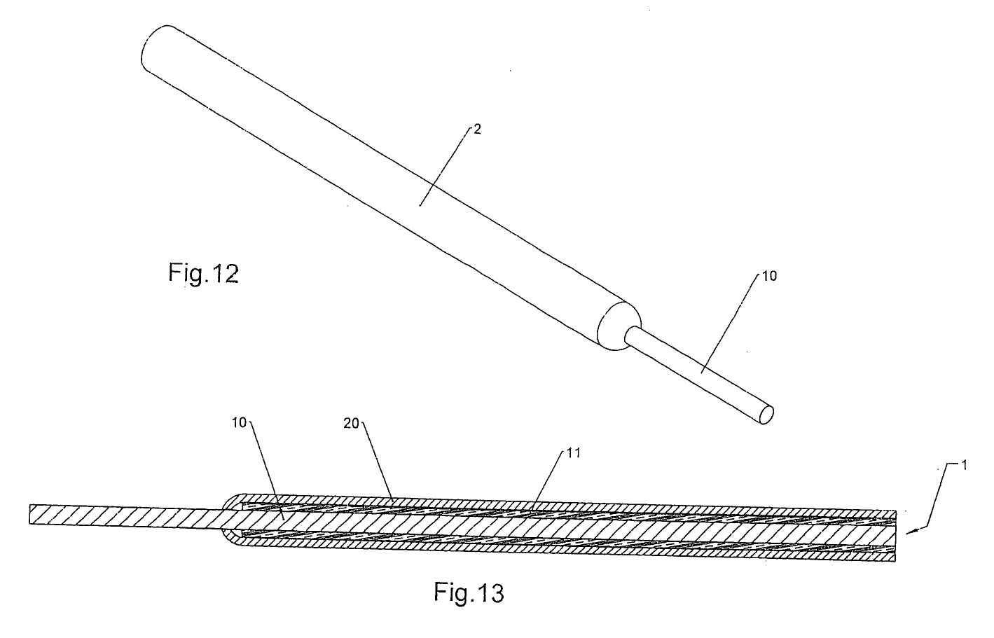

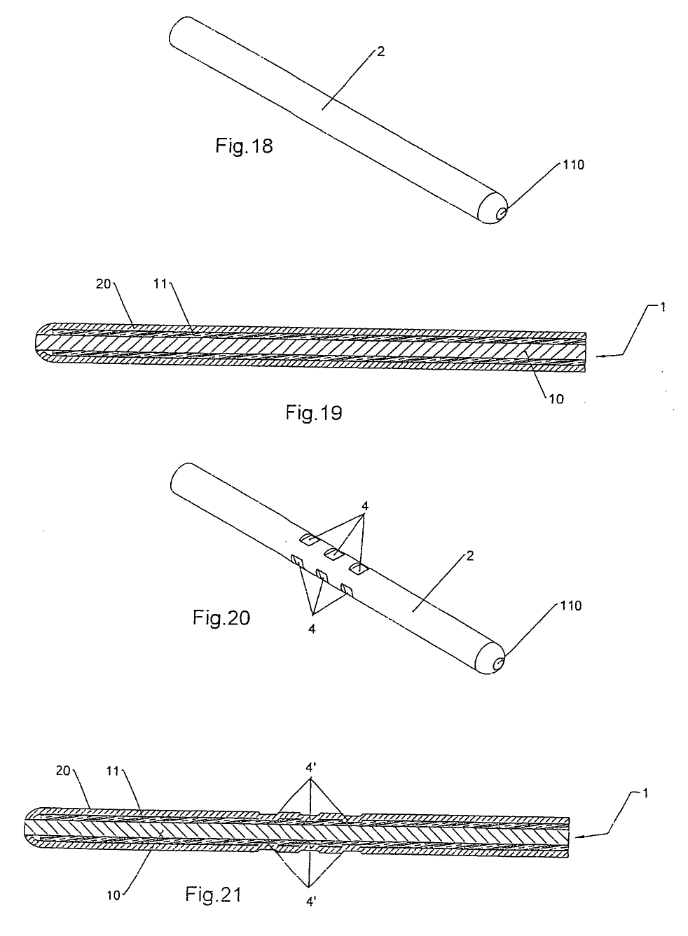

A first manufacturing procedure is illustrated in FIGS. 12 to 17 and a second manufacturing procedure is illustrated in FIGS. 18 to 21. These two procedures will be described in detail hereinafter. It should be noted that on these drawings, the illustrated cannula 2 and optical fibre 1 correspond to the embodiment of FIG. 3. The person skilled in the art will be able easily to transpose these procedures for the manufacture of the other embodiments of FIGS. 2, 4, 5 and 6.

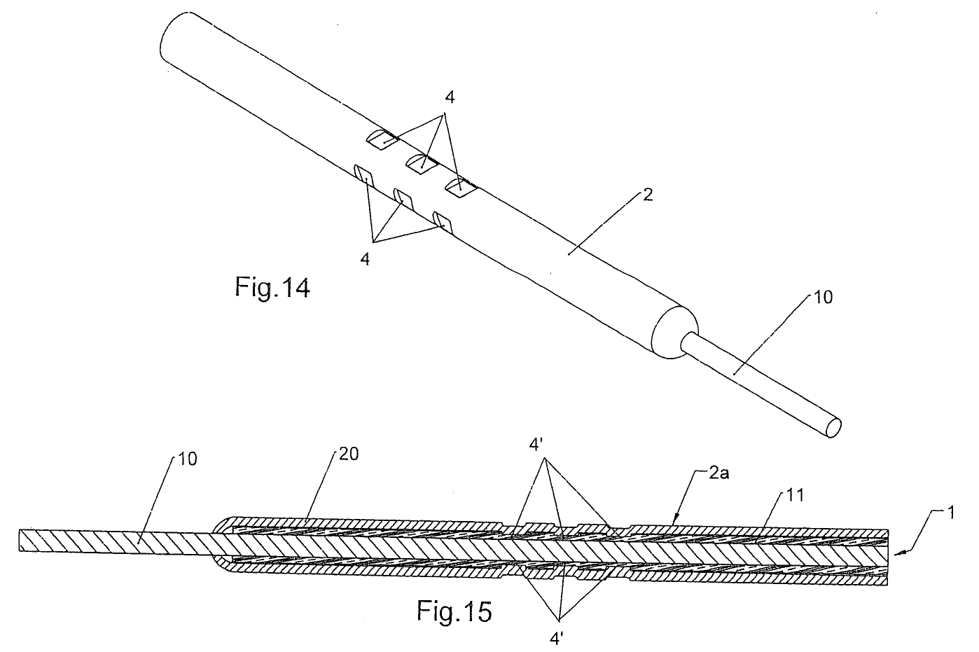

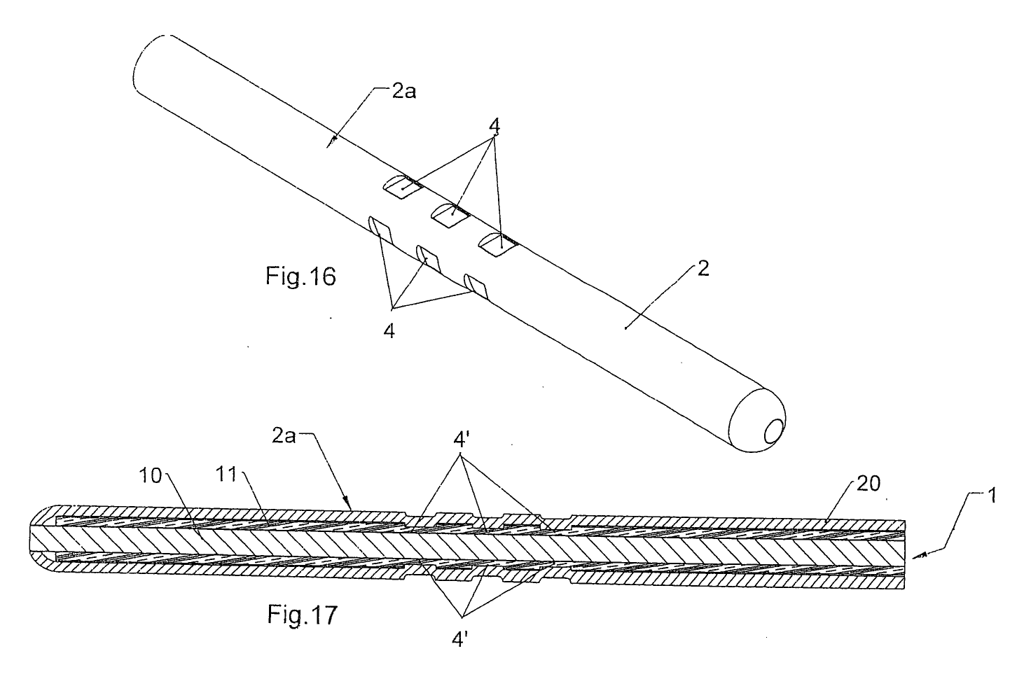

With reference to FIGS. 12 to 17, to manufacture the assembly E, in a first step (FIGS. 12 and 13), the cannula 2 is threaded into the optical fibre 1, the core 10 of which has been previously stripped on a distal portion of the optical fibre, until the protective sheath 11 of the fibre is brought into abutment in the cannula. For the embodiment of FIG. 4 or FIG. 5, the insert 23 will have been previously fixed to the hollow body 20, and the stripped distal part of the optical fibre is threaded into the insert 23 until the protective sheath 11 of the fibre is brought into abutment against the insert 23. In a second step (FIGS. 14 and 15), the cannula 2 is preferably crimped so as to solidly immobilise the fibre 1 with relation to the cannula 2. The crimping enables the wall of the cannula 2 and the protective sheath 11 to be locally deformed so as to immobilise the sheath 11, and thus the fibre 1, inside the cannula 2. Once the crimping has been carried out, radial deformations 4 are visible on the external wall 2a of the cannula 2. These radial deformations 4 on the external wall 2a of the cannula 2 result in deformations 4′ of the sheath 11, which enable the blocking of the fibre 1 within the cannula 2 and prevent it from sliding. Finally, in a third step (FIGS. 16 and 17), the core 10 of the optical fibre 1 is cut such that its distal extremity 101 is flush with the distal opening 210 of the cannula 2 [with the distal opening 232 of the insert 23 for the embodiment of FIG. 5].

A second method, shown in FIGS. 18 to 21, consists, in a first step (FIGS. 18 and 19), in stripping an appropriate length of the optical fibre 1 and in inserting the optical fibre 1 inside the cannula 2, such that the sheath 11 of the fibre is brought into abutment inside the cannula 2 and that the distal extremity 101 of the core 10 of the fibre 1 is flush with the distal opening 210 of the cannula [or the distal opening 232 of the insert 23 for the embodiment of FIG. 5]. Then, in a second step (FIGS. 20 and 21), the crimping of the cannula 2 onto the sheath 11 is carried out to immobilise the fibre 1 in the cannula 2.

In the two methods that have just been described, the crimping of the cannula 2 on the fibre 1 is carried out in a zone upstream from the zone of introduction of the assembly E into the human body, namely in a zone that is not intended to be introduced inside the human body.

In a sixth embodiment of the invention, shown in FIG. 7, the cannula 2 consists of a tubular body 20 with an external diameter D₂ and an internal diameter d₂. An additional electromagnetic radiation wave guide 3, called hereafter optical wave guide is fixed to the inside and in the distal part of the tubular body 20. This optical wave guide 3 is made out of any transparent material in the range of wavelengths of the laser source S, and in the illustrated example is flush with the distal opening 210 of the body 20 and blocks this distal opening 210.

The stripped distal part 100 of the core 10 of the optical fibre 1 is preferably positioned in abutment against this guide 3. The core 10 of the optical fibre, extended by the guide 3, forms a guiding means with the latter, enabling the electromagnetic radiation emitted by the laser source S to be guided by total internal reflections to the distal opening 210 of the body 20, and to emit frontally this electromagnetic radiation by the distal opening 210 of the cannula. In another embodiment, the distal extremity 101 of the stripped distal part 100 of the core 10 of the optical fibre 1 could thus be positioned in proximity to the optical wave guide 3 without however touching it; in this case, the distance between the distal extremity 101 of the core 10 of the optical fibre 1 and the optical wave guide 3 must be sufficiently small, such that the electromagnetic radiation, when leaving the core 10 of the fibre 1, is transmitted to the optical wave guide 3 without any significant loss.

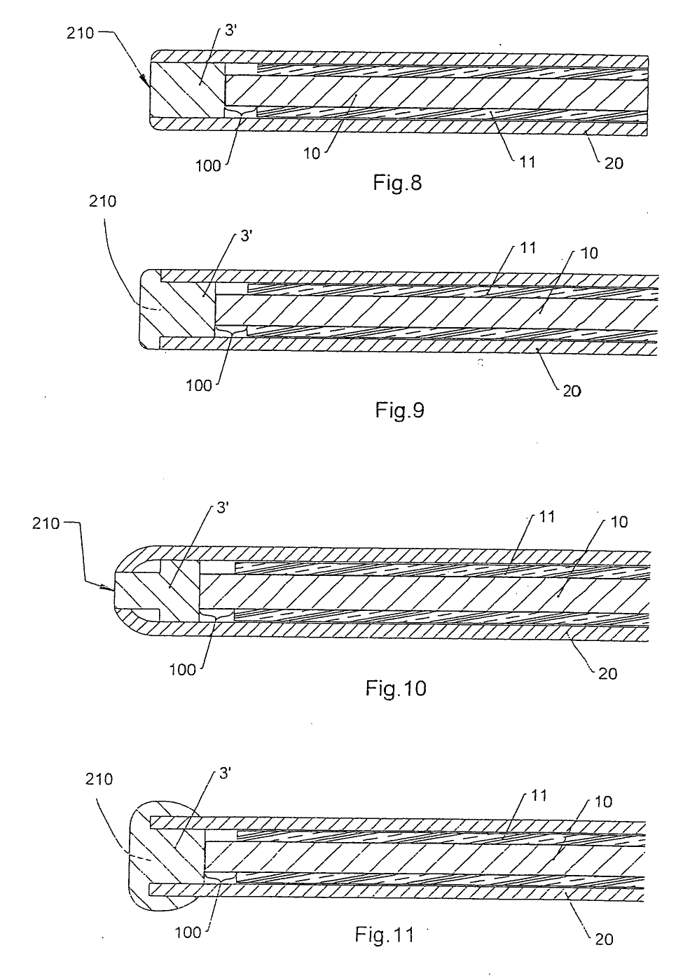

FIGS. 8 to 11 show other embodiments of the invention, which differ from the above-mentioned embodiment of FIG. 7 due to the implementation of optical wave guides 3′ displaying a different geometry to the optical wave guide 3 of FIG. 7.

The above-mentioned solution for immobilising the optical fibre 1 with relation to the cannula 2, through crimping the cannula 2 to the sheath 11 of the optical fibre 1, can also be implemented in the embodiments of FIGS. 7 to 11.

The invention is not restricted to the embodiments shown in the annexed drawings, which have been described only as examples. Other embodiments within the understanding of the person skilled in the art and covered by the annexed claims can be envisaged, without however falling outside the scope of the invention.

1. An assembly including a cannula, which comprises an opening at a so-called distal extremity and electromagnetic radiation wave guiding means, which include an optical fibre introduced inside the cannula and which enable the electromagnetic radiation to be guided to the opening of the distal extremity of the cannula, in such a way that this electromagnetic radiation is emitted frontally by said distal opening of the cannula, wherein said optical fibre includes a core surrounded by an external protective sheath, wherein the external diameter of the protective sheath of the optical fibre is essentially equal to the internal diameter of the cannula in at least one distal portion of said cannula, wherein the core of the optical fibre is stripped on a distal part of the fibre and the stripped distal part of the optical fibre is accommodated entirely inside the cannula.

2. The assembly according to claim 1, wherein the cannula includes in its distal part a hollow insert, which includes a cavity opened at both ends, and in that the stripped distal part of the core of the fibre is threaded into this hollow insert.

3. The assembly according to claim 1, wherein the sheath of the optical fibre is positioned in abutment inside the cannula.

4. The assembly according to claim 2, wherein the sheath of the optical fibre is positioned inside the cannula and in abutment against the insert.

5. The assembly according to claim 2, wherein the stripped distal part of the core of the fibre is accommodated entirely inside the hollow insert.

6. The assembly according to claim 2, wherein the hollow insert is made out of a thermally conductive material and enables the sheath of the optical fibre to be thermally protected.

7. The assembly according to claim 1, wherein the distal extremity of the core of the optical fibre is flush with the distal opening of the cannula.

8. The assembly according to claim 1, wherein the stripped distal part of the optical fibre blocks the distal opening of the cannula.

9. The assembly according to claim 1, wherein the electromagnetic radiation wave guiding means include an additional optical wave guide fixed to the cannula.

10. The assembly according to claim 9, wherein the additional optical wave guide is flush with the distal opening of the cannula.

11. The assembly according to claim 9, wherein the additional optical wave guide blocks the distal opening of the cannula.

12. The assembly according to claim 1, wherein the cannula is crimped onto the sheath of the optical fibre.

13. A laser instrument, wherein it includes an assembly described in claim 1, and a laser source coupled to the optical fibre of this assembly.
